(19) United States
(12) Patent Application Publication
(10) Pub. No.: US 2013/0053331 A1
(43) Pub. Date: Feb. 28, 2013

(54) SUGAR DERIVATIVES, METHODS FOR PREPARATION THEREOF, THEIR USE AS SURFACE-ACTIVE AGENTS

(71) Applicant: L'Air Liquide, Societe Anonyme pour l'Etude et ,'Exploitation des Procedes Georges Claude, Paris (FR)

(72) Inventors: Jerôme GUILBOT, Castres (FR); Hervé ROLLAND, Castres (FR); Daniel MULLER, Saint-Maur (FR)

(73) Assignee: L'Air Liquide, Societe Anonyme pour l'Etude et ,'Exploitation des Procedes Georges Claude, Paris (FR)

(21) Appl. No.: 13/662,895

(22) Filed: Oct. 29, 2012

Related U.S. Application Data
(63) Continuation of application No. 12/307,629, filed on Aug. 25, 2009.

(30) Foreign Application Priority Data
Jul. 10, 2006 (FR) 0652882

(51) Int. Cl. A61K 8/60

(52) U.S. Cl. 514/23

(57) ABSTRACT

Composition (C) comprising per 100% of its mass:
from 0.5% to 30% of one or more alcohols of formula (IV):

R1—OH (IV)

in which R1 represents an aliphatic radical having from 8 to 36 carbon atoms and
from 70% to 99.5% of one or more compounds of formula (I):

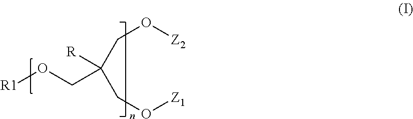

in which R represents an alkyl radical having from one to eight carbon atoms, n represents an integer greater than or equal to 1 and less than or equal to 10, R1 is as defined previously for formula (IV), at least one of the radicals Z₁ or Z₂ represents a radical:

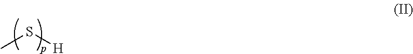

in which —S— represents the divalent residue of a reducing sugar and p represents a decimal number greater than or equal to 1 and less than or equal to 10 and the other one of the radicals Z₁ or Z₂ represents a hydrogen atom or said radical.

### CROSS REFERENCE TO RELATED APPLICATIONS

This is a continuation application of application Ser. No. 12/307,629, filed Aug. 25, 2009, which is a §371 of International PCT Application PCT/FR2007/051600, filed Jul. 5, 2007, the entire contents of which are incorporated herein by reference.

### FIELD OF THE INVENTION

The invention relates to novel surfactants obtained from sugars.

### BACKGROUND

The surface-active agents of the class of alkyl polyglycosides and alkenyl polyglycosides are now widely used as foaming agents, emulsifiers and/or solubilizers, either in cosmetic or pharmaceutical formulations or in formulations for industrial use.

U.S. Pat. No. 5,849,679 discloses nonionic surfactants comprising a mixture of anomeric α and β isomers of ethers of a glycoside compound with a monoether of glycerol or of polyglycerol and of C₁-C₂₂ alcohol and more particularly the following compounds: 1-O-n-decylglyceryl-D-glucopyranoside, 1-O-n-hexylglyceryl-D-glucopyranoside, 1-O-n-dodecyltriglyceryl-D-glucopyranoside, 1-O-n-oleyltriglyceryl-D-glucopyranoside and stearyl polyglyceryl-D-glucopyranoside.

German patent application DE 197 28 900 discloses the same type of nonionic surfactants derived from glycerol as those disclosed in the aforementioned document and more particularly the following compounds: lauryl glyceryl glucoside and (2-ethylhexyl) glyceryl glucoside.

French patent application 2 804 432 also discloses compounds derived from polyglyceryl glucosides as well as their use as surfactants.

### SUMMARY OF THE INVENTION

In the course of their research for the development of new surfactants, the inventors have developed novel glycosidic structures that have surface-active properties.

### DETAILED DESCRIPTION OF THE INVENTION

Thus, according to a first aspect, the invention relates to a composition (C) comprising, per 100% of its mass:
from 0.5% to 30% of one or more alcohols of formula (IV):

R1-OH (IV)
in which R1 represents a saturated or unsaturated, linear or branched aliphatic radical having from 8 to 36 carbon atoms, optionally substituted with one or more hydroxyl groups, and
from 70% to 99.5% of one or more compounds of formula (I):

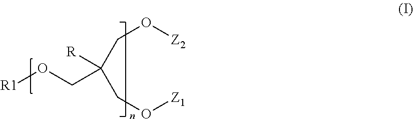

in which R represents a linear or branched alkyl radical having from one to eight carbon atoms optionally substituted with one or more hydroxyl radicals, n represents an integer greater than or equal to 1 and less than or equal to 10, R1 is as defined previously, at least one of the radicals Z₁ or Z₂ represents a radical:

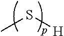

in which —S— represents the divalent residue of a reducing sugar and p represents a decimal number greater than or equal to 1 and less than or equal to 10 and the other one of the radicals Z₁ or Z₂ represents a hydrogen atom or said radical

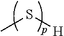

“Saturated or unsaturated, linear or branched aliphatic radical having from 8 to 36 carbon atoms, optionally substituted with one or more hydroxyl groups, and/or one or more oxo groups”, designates for R1, in formulas (I) and (IV) as defined above:
the linear alkyl radicals, for example the octyl, decyl, dodecyl, tetradecyl, hexadecyl, octadecyl, eicosyl or docosyl radicals;
the radicals obtained from the isoalkanols of formula (A):

(CH₃)(CH₃)CH—(CH₂)ₘ—CH₂—OH (A)
in which m represents an integer between 4 and 18, for example isooctyl, isodecyl, isododecyl, isotetradecyl, isohexadecyl or isooctadecyl radicals;
the 2-ethylhexyl radical or the branched alkyl radicals obtained from the Guerbet alcohols of formula (B):

CH(CₛH₂ₛ₊₁)(CₜH₂ₜ₊₁)—CH₂—OH (B)
in which t is an integer between 4 and 18, s is an integer between 2 and 16 and the sum s+t is greater than or equal to 8, for example the 2-ethyldecyl, 2-butyloctyl, 2-ethyldodecyl, 2-butyldecyl, 2-hexyloctyl, 2-ethyltetradecyl, 2-butyldecyl, 2-hexyloctyl, 2-ethylhexadecyl, 2-butyltetradecyl, 2-hexyldodecyl, 2-octyldecyl, 2-hexyldecyl, 2-octyldodecyl, 2-decyltetradecyl, 2-dodecylhexadecyl, 2-tetradecyloctadecyl, 2-tetradecyleicosyl, 2-hexadecyloctadecyl or 2-hexadecyleicosyl radicals; or the radicals obtained from the homologues of Guerbet alcohols, for example the 2-propylheptyl radical.
the unsaturated linear radicals such as the undecenyl, docecenyl, tetradecenyl, hexadecenyl, octadecenyl, octadecadienyl, octadecatrienyl, octadecatetraenyl, eicosenyl, eicosadienyl or docosenyl radicals, for example the 10-undecenyl, 4-dodecenyl, dodecenyl, oleyl, isooleyl, linoleyl, linolenyl or 10,13-eicosadienyl unsaturated radicals;
the saturated or unsaturated, linear or branched aliphatic radicals having from 8 to 36 carbon atoms substituted with 1 or two hydroxyl groups, such as the hexadecyl, hydroxyoctadecyl, dihydroxydocosyl or dihydroxy-octadecyl hydroxyl radicals, for example the 3-hydroxyhexadecyl radicals, 4-hydroxyhexadecyl acid, 11-hydroxyhexadecyl acid, 16-hydroxyhexadecyl, 12-hydroxystearyl or 8,9-dihydroxystearyl acid.

Linear or branched alkyl radical having from one to eight carbon atoms optionally substituted with one or more hydroxyl radicals or one or more”, denotes notably for R in formula (I) as defined above, the methyl, ethyl, propyl, isopropyl, butyl, isobutyl, pentyl, isopentyl, hexyl, isohexyl, heptyl, isoheptyl, octyl, isooctyl, hydroxymethyl or 2-hydroxyethyl radicals.

Residue of a reducing sugar” denotes principally for —S— in formula (I) as defined above, the glucose, sucrose, fructose, idose, galactose, maltose, maltotriose, lactose, cellobiose, mannose, xylose, arabinose, ribose, dextran or tallose residues.

In formula (I) as defined above, p is a decimal number, which represents the average degree of polymerization of the residue S.

When p is an integer, (S)ₚ is the polymeric residue of class p of the residue S.

When p is a decimal number, formula (I) represents a mixture of compounds a₁ [(Q)(S)]+a₂[(Q)(S)₂]+a₃[(Q)(S)₃]⁺. . . +a_{q} [(Q)(S)_{q}] in which Q represents the group bound to the sugar residue(s) S of formula (I) with q representing an integer between 1 and 10 and in molar proportions a₁, a₂, a₃, . . . a_{q} such that:

q=1

Σaₚ=1;a₁>0

q=10

In the definition of formula (I) as defined previously, n is an integer that represents the degree of polymerization of the trivalent radical:

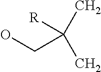

Thus, when n is for example equal to two, it is the following tetravalent radical:

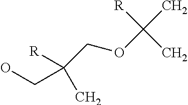

in all its stereoisomeric forms.

When n is for example equal to three, it is either the following pentavalent radical:

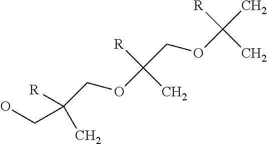

in all its stereoisomeric forms;
or the following pentavalent radical:

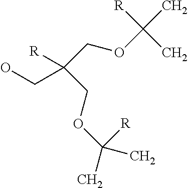

in all its stereoisomeric forms.

According to a first particular aspect of the present invention, in formula (IV), R1 represents a radical selected from the octyl, decyl, dodecyl, tetradecyl, hexadecyl, octadecyl, eicosyl, 2-ethylhexyl, 2-butyloctyl, 2-butyldecyl, 2-hexyloctyl, 2-hexyldecyl, 2-hexyldodecyl, 2-octyldecyl, 2-octyldodecyl, 2-decyltetradecyl, isododecyl, isotetradecyl, isohexadecyl, isooctadecyl, 10-undecenyl, oleyl, isooleyl, linoleyl, linolenyl or 10,13-eicosadienyl, 12-hydroxystearyl or 2-propylheptyl radicals.

According to a second particular aspect of the present invention, in formula (I), —S-represents a divalent residue of glucose or of xylose.

According to a third particular aspect of the present invention, in formula (I), p is between 1.005 and 5 and more particularly between 1.05 and 2.

According to a fourth particular aspect of the present invention, in formula (I), R is selected from the methyl, ethyl, propyl, butyl, pentyl, hexyl, heptyl, octyl and hydroxymethyl radicals.

The invention also relates to a method of preparation of a composition (C) as defined previously, comprising the following successive stages:

A stage a) of reaction of an alcohol or of a mixture of alcohols of formula (IV):

R1-OH (IV)

in which R1 represents a saturated or unsaturated, linear or branched aliphatic radical having from 8 to 36 carbon atoms, optionally substituted with one or more hydroxyl groups or a mixture of alcohols of formula (IV), with a 3,3-disubstituted oxetane of formula (V):

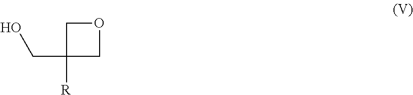

in which R represents a linear or branched alkyl radical having from one to eight carbon atoms, optionally substituted with one or more hydroxyl radicals, leading to a mixture (M) comprising the alcohol of formula (IV) that has not reacted and one or more compounds of formulas (II):

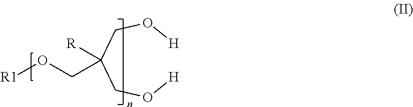

in which R and R1 are as defined previously and n represents an integer greater than or equal to 1 and less than or equal to 10;

A stage b) of reaction of said mixture (M) with a reducing sugar of formula (III):

S—H (III)

to obtain said surface-active composition (C).

In the method as defined above, stage (a) is generally carried out in the presence of a Lewis acid as catalyst; we may mention for example, boron trifluoride stabilized in diethyl ether (BF₃.Et₂O), tin tetrachloride (SnCl₄), titanium tetrachloride (SnTi₄) or aluminium trichloride (AlCl₃).

Certain compounds of formula (V) are known compounds; 3-ethyl-3-(hydroxymethyl) oxetane of formula (V) (or TMP oxetane) is a commercial product identified with the numbers RN 3047-32-3 and EINECS 221-54-0.

In the method as defined above, stage (b) is generally carried out in the presence of a strong acid as catalyst, for example sulphuric acid, hydrochloric acid, phosphoric acid, methanesulphonic acid, p-toluenesulphonic acid, trifluoromethanesulphonic acid or hypophosphorous acid.

Composition (C) according to the present invention, when it is obtained by the method as defined previously, can additionally contain up to 5% of its mass of residual products, principally the reducing sugar of formula (III), the oxetane derivative of formula (V) and/or compounds of formula (VI):

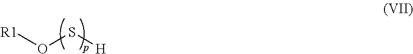

in which R1, S and p are as defined previously, and water.

According to a particular aspect of the method as defined above, it additionally comprises a stage (c) of dehydration of a compound of formula (VII):

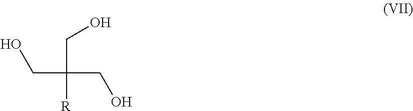

in which R is as defined previously, to obtain the compound of formula (V).

Stage (c) as defined above is generally composed of an initial stage of reaction of the compound of formula (VII) with a dialkyl carbonate, for example dimethyl carbonate, diethyl carbonate or dibutyl carbonate, to obtain a cyclic carbonate of the derivative of formula (VII), followed by decarboxylation of said cyclic carbonate to obtain the disubstituted oxetane of formula (V).

Examples of known compounds of formula (VII) are trimethylolpropane (TMP) (R representing an ethyl radical) or pentaerythritol (R representing a hydroxymethyl radical).

When in formula (II) as defined above, n is equal to 1, said formula (II) is representative of the compound of the following formula (IIa):

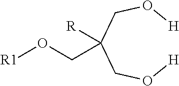

An example of a compound of formula (IIa) is the compound of formula (IIa1):

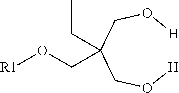

or that of formula (IIa2)

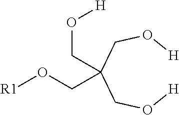

corresponding to formula (IIa) in which R represents respectively an ethyl radical or a hydroxymethyl radical; said compounds of formulas (II) being in all possible stereoisomeric forms with respect to the quaternary carbon whether it is the isomer (R), the isomer (S) or the racemate (RS).

When in formula (II), n is equal to 2, said formula (II) is representative of the compound of the following formula (IIb):

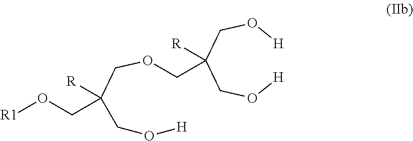

said compound of formula (IIb) being in all possible stereoisomeric forms with respect to its quaternary carbon whether it is the isomers (R), (S) or racemates thereof.

When in formula (II), n is equal to 3, said formula (II) is representative of the compound of the following formula (IIc1):

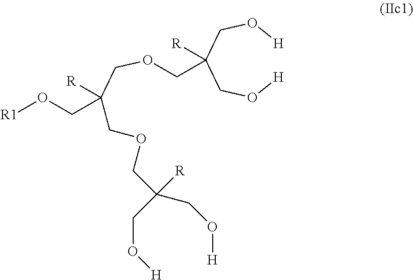

or of the compound of formula (IIc2):

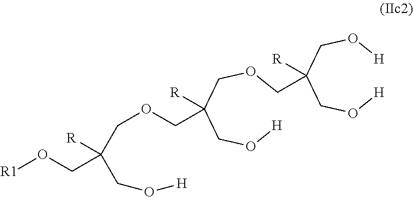

said compounds of formulas (IIc1) and (IIc2) being in all possible stereoisomeric forms.

The invention also relates to a compound of formula (Ia):

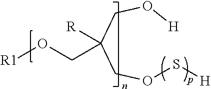

corresponding to formula (I) as defined previously in which one of the radicals Z₁ or Z₂ represents a hydrogen atom and the other represents the radical:

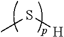

The invention relates more particularly to the compound of formula (Ia1):

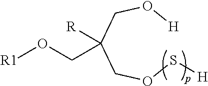

corresponding to formula (Ia) as defined previously, in which n is equal to 1, and more particularly the compound of formula (Ia3):

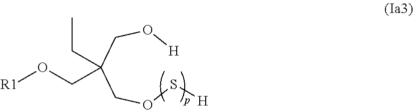

or a compound of formula (Ia4):

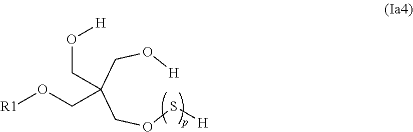

corresponding to formula (Ia1) as defined above, in which R represents respectively an ethyl radical or a hydroxymethyl radical, said compounds of formulas (Ia1), (Ia3) and (Ia4) being in all possible stereoisomeric forms.

When in formula (Ia), n is equal to 2, said formula (Ia) is principally representative of the compound of formula (Ib1):

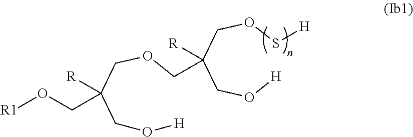

or of the compound of formula (Ib2):

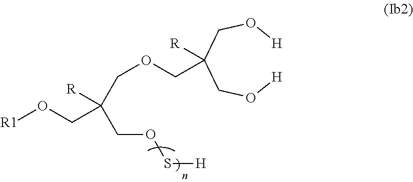

said compounds of formulas (Ib1) and (Ib2) being in all possible stereoisomeric forms.

The invention also relates to a compound of formula (Ic):

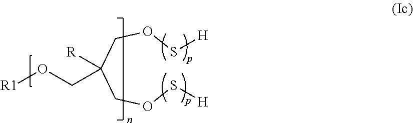

corresponding to formula (I) as defined previously in which each of the radicals Z₁ or Z₂ represents the radical:

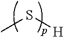

The invention also relates to the use of a composition (C) or of a compound of formula (Ia) as defined previously, as surfactant and more particularly as foaming agent, emulsifier, wetting agent, dispersant or detergent.

The invention finally relates to a cosmetic or pharmaceutical composition, characterized in that it contains, as surfactant, a composition (C) or at least one compound of formula (Ia), as defined previously.

The following experimental section illustrates the invention, though without limiting it.

### Example 1

Preparation of a composition C1 for which, in formulas (I) and (IV), R1 represents the octadecyl radical and, in formula (I), R represents the ethyl radical and S represents the glucose residue

### Stage a): Opening of TMP Oxetane by Octadecanol:

TMP oxetane is the compound of formula (V) as defined previously, in which R represents the ethyl radical. This first stage is carried out at 120° C. in the presence of a catalytic amount of Lewis acid (BF₃.Et₂O). Two different stoichiometries in TMP oxetane were investigated (tests 1 and 2), the first involving an equimolar quantity relative to the octadecanol and the second involving an excess of 3 molar equivalents, also relative to the octadecanol. The operating conditions are as follows: Melting of octadecanol at ˜120° C. under nitrogen atmosphere, addition of 0.5% of BF₃.Et₂O (relative to the total mass of the reactants) with mechanical stirring, then gradual addition (in about 180 minutes) of the TMP oxetane, holding at 120° C. for 90 to 180 minutes, then cooling. The characteristics of the mixtures (M1) and (M2) obtained are presented in the following Table 1a (RT: room temperature; IA: Acid number; IOH: Hydroxyl number).

TABLE 1a

| | Test 1 | Test 2 |
|---|---|---|
| | (M1) | (M2) |
| Stoichiometry TMP oxetane/C-18 OH | 1/1 | 3/1 |
| Appearance at RT | White | White |
| | solid | solid |
| pH at 1% in water/isopropanol (50/50) | 5.0 | 4.9 |
| Water (wt. %) - Standard NFT 73201 | 0.01 | 0.03 |
| Melting point (° C.) - Kofler bench | <44 | <44 |
| IA (mg KOH/g) Standard NFT 60-204 | 2.2 | 1.4 |
| IOH (mg KOH/g) Standard USP (XXI NF XVI | 285.6 | 351.3 |
| Jan. 1, 1985) | | |
| Octadecanol (wt. %) | 32.0 | 12.3 |
| TMP oxetane (wt. %) | 0.4 | 3.4 |
| Monoadduct (compound of formula (IIa1) with R1: | 27.8 | 18.2 |
| octadecyl) (wt. %) | | |
| Diadduct (compound of formula (II) with R1: | 15.9 | 15.5 |
| octadecyl, R: ethyl and n = 2) (wt. %) | | |
| Triadduct (compound of formula (II) with R1: | 9.7 | 14.7 |
| octadecyl, R: ethyl and n = 3) (wt. %) | | |
| Tetraadduct (compound of formula (II) with R1: | 4.8 | 11.4 |
| octadecyl, R: ethyl and n = 4) (wt. %) | | |
| Pentaadduct (compound of formula (II) with R1: | 1.8 | 8.9 |
| octadecyl, R: ethyl and n = 5) (wt. %) | | |
| Hexaadduct (compound of formula (II) with R1: | Not | 6.6 |
| octadecyl, R: ethyl and n = 6) (wt. %) | detected | |
| Heptaadduct (compound of formula (II) with R1: | Not | 4.2 |
| octadecyl, R: ethyl and n = 7) (wt. %) | detected | |
| Octaadduct (compound of formula (II) with R1: | Not | 1.4 |
| octadecyl, R: ethyl and n = 8) (wt. %) | detected | |

In general, the results demonstrate good reactivity between octadecanol and TMP oxetane. Theoretically, the hydroxyl number of the reaction mixture should not vary during the reaction and the final measured values are consistent with those calculated (Mixture M1: 285.6 mg KOH/g for a theoretical value of 289.2 mg KOH/g; Mixture M2: 351.3 mg KOH/g for a theoretical value of 361.8 mg KOH/g). Quantitative determinations of residual octadecanol show, respectively, that in tests 1 and 2, respectively, about 54% and 72% of the starting octadecanol was consumed during the reaction. Quantitative determinations of residual TMP oxetane show respectively that, in tests 1 and 2, more than 98% and 94% of the starting TMP oxetane had reacted. Finally, the distribution of the various adducts, established by gas chromatography, after normalization to 100%, shows that the introduction of an excess of TMP oxetane leads to a higher consumption of octadecanol and promotes the phenomenon of polymerization of the TMP unit (higher n).

Stage b): Acetalization of the TMP Oxetane/Octadecanol Adducts with Glucose:

The procedure adopted was as follows: Melting of mixtures M1 and M2 at about 100° C. at atmospheric pressure and under nitrogen atmosphere, then addition, with mechanical stirring, of a molar equivalent of anhydrous glucose (the amount is calculated from the number of moles of octadecanol introduced in the first stage), addition of 1% of 98% sulphuric acid (wt. % expressed relative to the mass of glucose introduced), gradual vacuum [about 30 10² Pa (30 mbar)] and acetalization with glucose for 6 hours at about 100° C.; cooling to 80° C. and neutralization by adding a 30% solution of soda lye to obtain pH close to 7; hot filtration on a filter plate with porosity of 4 micrometers and conditioning. The analytical characteristics of the two compositions (C1) and (C2) obtained are presented in Table 1b below.

TABLE 1b

| | Composition | Composition |
|---|---|---|
| Analyses | C1 | C2 |
| Starting product | Mixture M1 | Mixture M2 |
| Appearance at RT | White solid | Beige solid |
| pH at 1% in water/ethanol (50/50) | 7.8 | 7.6 |
| Water (%) - NFT 73201 | 0.41 | 0.42 |
| Color of molten product - NFT 20-030 (vcs) | 2.0 | Not |
| | | measurable |
| Melting point (° C.) Kofler bench | ~50° C. | 55 65° C. |
| IA (mg KOH/g) - NFT 60-204 | 2.0 | 1.0 |
| IOH (mg KOH/g) - USP XXI NF XVI | 393.9 | 412.8 |
| Jan. 1, 1985 | | |
| Octadecanol (wt. %) | 24.2% | 9.0% |
| TMP oxetane (wt. %) | <0.05% | <0.05 |
| Glucose (wt. %) | 0.2% | <0.1 |
| Compounds of formula (VI) R1: octadecyl; | 3.0% | 1.0% |
| S: glucose (wt. %) | | |
| Compounds of formula (I) R1: octadecyl; S: | 3.6% | 1.6% |
| glucose; R: ethyl n = 1 (wt. %) | | |
| Compounds of formula (I) R1: octadecyl; S: | >65% | >85% |
| glucose; R: ethyl n > 1 (wt. %) | | |

The significant increase in hydroxyl number in the course of the reactions indicates satisfactory reactivity between the glucose and the “octadecanol+TMP” adducts.

### Example 2

Preparation of two compositions C3 and C4 for which, in formulas (I) and (IV), R1 represents the octadecyl and hexadecyl radical and in formula (I) R represents the ethyl radical and S represents the glucose residue.

The two stages developed in the preceding example are employed, replacing the octadecanol with cetearyl alcohol (50/50 molar mixture of hexadecanol and octadecanol) in two tests with the stoichiometric ratios glucose/alcohol of formula (IV) TMP oxetane. The analytical characteristics of the two compositions (C3) and (C4) obtained are presented in Table 2 below.

TABLE 2

| | Composition | Composition |
|---|---|---|
| Analyses | C3 | C4 |
| Starting product | Mixture M3 | Mixture M3 |
| Molar stoichiometry alcohol of formula | 1/2.5 | 1/2.5 |
| (IV)/TMP oxetane (formula (V) | | |
| Molar stoichiometry glucose/alcohol of | 0.5/1 | 1/1 |
| formula (IV) | | |
| Appearance at RT | Beige solid | Yellow solid |
| pH at 1% in water/isopropanol | 6.6 | 6.0 |
| Water (%) - NFT 73201 | 0.17 | 0.27 |
| Color of molten product - NFT 20-030 | 2.0 | 2 |
| (vcs) | | |
| Melting point (° C.) Kofler bench | <44° C. | <44° C. |
| IA (mg KOH/g) - NFT 60-204 | 1.3 | 1.3 |
| IOH (mg KOH/g) - USP XXI NF XVI | 395.1 | 427.5 |
| Jan. 1, 1985 | | |
| Cetearyl alcohol (C16/C18) (wt. %) | 10.6% | 9.8% |
| TMP oxetane (water. %) | <0.01% | <0.01% |
| Glucose (wt. %) | <0.1% | 0.4% |
| Compounds of formula (VI) R1: | 0.8% | 1.0% |
| cetearyl; S: glucose (wt. %) | | |
| Compounds of formula (I) R1: cetearyl; | >85% | >85% |
| S: glucose; R: ethyl, n > 1 (wt. %) | | |

### Example 3

### Evaluation of Emulsifying Properties

### 3.1—For Water-in-Oil Emulsions:

Three types of oil were used: a paraffin oil (MARCOL™ 52), a polar oil (C-8/10 Triglyceride 5545) and an aromatic oil (SOLVESSO™ 100). The two formulations adopted are:
20% of oil, 2% of surface-active composition and 78% of tap water+1% MgSO₄,
50% of oil, 5% of surface-active composition and 45% of tap water+1% MgSO₄.

The procedure for preparation of the emulsion comprises the following stages:
1—Mixing of the oil and the surface-active composition, with homogenizing with a spatula, in a 400-ml-tall beaker, on a water bath at 80° C.;
2—Addition of the aqueous phase (tap water+1% of dry MgSO₄ (2% of MgSO₄ heptahydrate)) at 80° C.;
3—Shearing of the mixture in a Silverson™ (8000 rev/min for 4 min; rotor/stator as low as possible in the beaker, and gently turning the beaker) and
4—Cooling to room temperature while stirring gently to obtain 200 g of emulsion.

The results for stability at room temperature (RT) and at 40° C. are presented in Table 3 below. Cases when the mixtures are no longer completely emulsified after 24 hours at room temperature are not included.

TABLE 3

| | | | Observations | | |
|---|---|---|---|---|---|
| | | | | t = 13 days (mixture M1 | |
| | | | | and composition C2) | |
| | | formulation: | | t = 42 days (composition | |
| | | Composition | t = 1 day | C1 and mixture M2) | |
| | | Oil/(Water + | Room | Room | |
| Composition | Oil | MgSO₄) | temperature | temperature | 40° C. |
| Mixture M1 | Triglyceride | 5/50/45 | Em.: 100% | Em.: 93% | Em.: 71% |
| | 5545 | | | | |
| Mixture M1 | MARCOL ™ | 5/50/45 | Em.: 100% | Em.: 80% | Em.: 51% |
| | 52 | | | | |
| Mixture M2 | MARCOL ™ | 5/50/45 | Em.: 100% | Em.: 90% | Em.: 76% |
| | 52 | | | | |
| Composition C1 | Triglyceride | 2/20/78 | Em: 100% | Em.: 100% | Em.: 47% |
| | 5545 | | | | |
| Composition C1 | Triglyceride | 5/50/45 | Em.: 100% | Em.: 100% | Em.: 90% |
| | 5545 | | | | |
| Composition C2 | SOLVESSO ™ | 5/50/45 | Em.: 100% | Em.: 38% | 3 phases |
| | 100 | | | | |

Em.: corresponds to the volume of phases emulsified relative to the initial mass prepared; in fact, Em. 93% signifies that a volume representing 7% of the initial volume of emulsion has separated.

The stabilities observed show an emulsifying capacity of the compositions according to the invention at room temperature and at D1 for the fatty phases investigated. C1 possesses good emulsifying capacity for preparing water-oil emulsions with triglyceride 5545.

### 3.2—For Oil-in-Water Emulsions:

In the case of oil-in-water emulsions, two types of oil were used: a paraffin oil (MARCOL™ 52) and a polar oil (C8/C10 Triglyceride 55). The formulations adopted are identical to those previously described, using MgSO₄-free tap water.

The procedure for preparing the emulsion comprises the following stages:

1—Mixing of the tap water and the surface-active composition at 80° C.,
2—Addition of the oil and stirring with a spatula,
3—Shearing of the mixture in a Silverson (8000 rev/min for 4 min; rotor/stator as low as possible in the beaker, gently turning the beaker) and
4—Cooling to room temperature while stirring gently to obtain 200 g of emulsion.

The results for stability at room temperature and at 40° C. are presented in Table 4 below. Cases when the mixtures are no longer completely emulsified after 24 hours at room temperature are not included.

TABLE 4

| | | | Observations | | |
|---|---|---|---|---|---|
| | | | | t = 13 days | |
| | | | | (composition C2) | |
| | | | | t = 42 days | |
| | | Formulation: | | (composition C1 and | |
| | | Composition/ | t = 1 day | MONTANOV ™ 68) | |
| Composition | Oil | Oil/Water | RT | RT | 40° C. |
| MONTANOV ™ | Triglyceride | 2/20/78 | Em.: 100% | Em.: 100% | Em.: 100% |
| 68 | 5545 | | | | |
| MONTANOV ™ | Triglyceride | 5/50/45 | Em.: 100% | Em.: 100% | Em.: 100% |
| 68 | 5545 | | | | |
| MONTANOV ™ | Triglyceride | 0.8/21.2/78 | Em.: 66% | Phase | Phase |
| 68 | 5545 | | | separation | separation |
| MONTANOV ™ | Triglyceride | 0.3/21.7/78 | Em.: 67% | Phase | Phase |
| 68 | 5545 | | | separation | separation |
| Composition C1 | Triglyceride | 2/20/78 | Em.: 100% | Em.: 100% | Em.: 100% |
| | 5545 | | | | |
| Composition C1 | Triglyceride | 5/50/45 | Em.: 100% | Em.: 100% | Em.: 100% |
| | 5545 | | | | |
| Composition C1 | MARCOL ™ | 2/20/78 | Em.: 100% | Em.: 100% | Em.: 87% |
| | 52 | | | | |
| Composition C2 | Triglyceride | 2/20/78 | Em.: 100% | Em.: 100% | Em.: 100% |
| | 5545 | | | | |
| Composition C2 | Triglyceride | 5/50/45 | Em.: 100% | Em.: 100% | Em.: 100% |
| | 5545 | | | | |
| Composition C2 | MARCOL ™ | 2/20/78 | Em.: 100% | Em.: 100% | Em.: 88% |
| | 52 | | | | |
| Composition C2 | MARCOL ™ | 5/50/45 | Em.: 100% | Em.: 79% | 3 phases |
| | 52 | | | | |

Overall, the glucosides of “octadecanol+TMP” adduct display remarkable emulsifying capacity for oil in water O/W in comparison with Montanov™ 68 (mixture of compounds of formula (IV) and (VI) with R1 of C16-C18).

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A composition (C) comprising per 100% of the composition's mass:
from 0.5% to 30% of one or more alcohols of formula (IV):
R1-OH (IV)
in which R1 represents a saturated or unsaturated, linear or branched aliphatic radical having from 8 to 36 carbon atoms, and
from 70% to 99.5% of one or more compounds of formula (I) selected from: 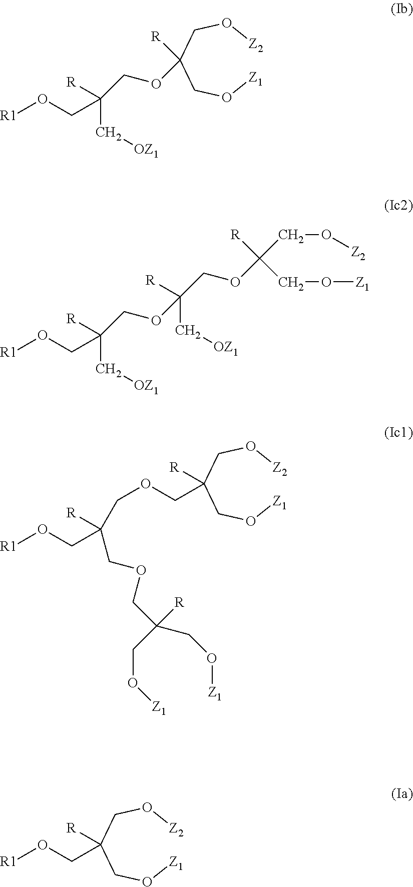
where R represents a linear or branched alkyl radical having from one to eight carbon atoms, R1 is as defined previously for formula (IV), at least one of the radicals Z₁ or Z₂ represents a radical: 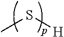
in which —S— represents the divalent residue of a reducing sugar and p represents a decimal number greater than or equal to 1 and less than or equal to 10 and the other one of the radicals Z₁ or Z₂ represents a hydrogen atom or said radical:

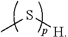

2. The composition (C) as defined in claim 1, for which R1 represents a radical selected from the radicals octyl, decyl, dodecyl, tetradecyl, hexadecyl, octadecyl, eicosyl, 2-ethylhexyl, 2-butyloctyl, 2-butyldecyl, 2-hexyloctyl, 2-hexyldecyl, 2-hexyldodecyl, 2-octyldecyl, 2-octyldodecyl, 2-decyltetradecyl, isododecyl, isotetradecyl, isohexadecyl, isooctadecyl, 10-undecenyl, oleyl, isooleyl, linoleyl, linolenyl or 10,13-eicosadienyl, 12-hydroxystearyl and 2-propylheptyl.

3. The composition (C) as defined in claim 1, wherein —S— represents a divalent glucose residue.

4. The composition (C) as defined in claim 1, for which in formula (I), —S-represents a divalent xylose residue.

5. The composition (C) as defined in claim 1, wherein p is between 1.005 and 5.

6. The composition (C) as defined in claim 1, wherein R is selected from the methyl, ethyl, propyl, butyl, pentyl, hexyl, heptyl, octyl and hydroxymethyl radicals.

7. A method of preparation of a composition (C) as defined in claim 1, comprising the following successive steps of chemical reactions:
a step a) of reaction of an alcohol of formula (IV):
R1-OH (IV)
in which R1 represents a saturated or unsaturated, linear or branched aliphatic radical having from 8 to 36 carbon atoms, or of a mixture of alcohols of formula (IV), with a 3,3-disubstituted oxetane of formula (V): 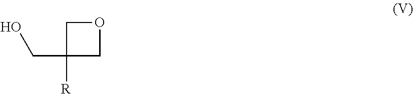
in which R represents a linear or branched alkyl radical having from one to eight carbon atoms, leading to a mixture (M) comprising a residual alcohol of formula (IV) that had not reacted and one or more compounds chosen from the compounds of formula (IIa), (IIb), (IIc1) or (IIc2): 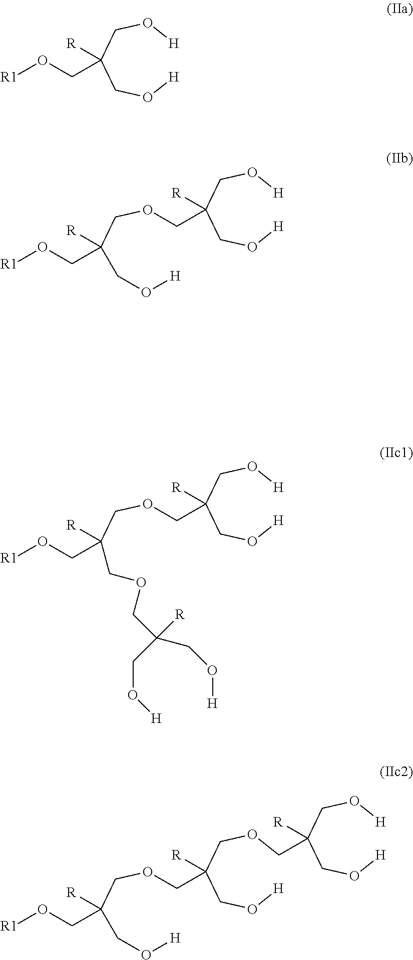
in which R and R1 are as defined previously;
a step b) of reaction of said mixture (M) with a reducing sugar of formula (III):
S—H (III)
to obtain said composition (C).

8. The method as defined in claim 7, additionally comprising a step c) of dehydration of a compound of formula (VII): 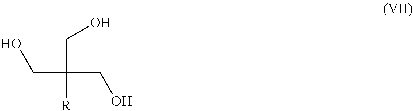
in which R represents a linear or branched alkyl radical having from one to eight carbon atoms, to obtain the compound of formula (V).

9. The composition (C) as defined in claim 1, wherein p is between 1.05 and 2.

10. A cosmetic or pharmaceutical composition, comprising the composition (C) of claim 1.

11. The composition (C) of claim 1, wherein R1 represents a saturated or unsaturated, linear or branched aliphatic radical having from 8 to 36 carbon atoms and is substituted with one or more hydroxyl groups.

12. The composition (C) of claim 1, wherein R represents a linear or branched alkyl radical having from one to eight carbon atoms and is substituted with one or more hydroxyl radicals.

13. The method of claim 7, wherein R1 represents a saturated or unsaturated, linear or branched aliphatic radical having from 8 to 36 carbon atoms and is substituted with one or more hydroxyl groups,

14. The method of claim 7, wherein R represents a linear or branched alkyl radical having from one to eight carbon atoms and is substituted with one or more hydroxyl radicals.
